(19) United States
(12) Patent Application Publication
(10) Pub. No.: US 2022/0003645 A1
(43) Pub. Date: Jan. 6, 2022

(54) KITS AND METHODS FOR ISOLATING PROTEIN FROM BIOLOGICAL AND ENVIRONMENTAL SAMPLES

(71) Applicant: QIAGEN Sciences, LLC, Germantown, MD (US)

(72) Inventors: Heather Callahan, Escondido, CA (US); Suzanne Kennedy, Houston, TX (US); Mark Brolaski, Encinitas, CA (US)

(21) Appl. No.: 17/387,019

(22) Filed: Jul. 28, 2021

Related U.S. Application Data
(62) Division of application No. 14/642,556, filed on Mar. 9, 2015, now abandoned.
Application No. 14/642,556 is a continuation of application No. PCT/US2014/032995, filed on Apr. 4, 2014.
(60) Provisional application No. 61/809,237, filed on Apr. 5, 2013.

(51) Int. Cl. G01N 1/40; C07K 1/14; C12N 15/10; C12Q 1/6806

(52) U.S. Cl. CPC G01N 1/4055; C12Q 1/6806; C12N 15/1003; C07K 1/145

(57) ABSTRACT

Provided are methods and compositions for isolating protein or other biomolecules from biological or environmental samples. The isolated biomolecules are substantially free of contaminants.

### RELATED APPLICATION DATA

This application is a divisional of U.S. application Ser. No. 14/642,556 filed Mar. 9, 2015, which is a continuation of International Application No. PCT/US2014/032995 filed Apr. 4, 2014, which claims benefit of U.S. Application Ser. No. 61/809,237 filed Apr. 5, 2013. U.S. application Ser. No. 14/642,556 is herein incorporated by reference in its entirety.

### TECHNICAL FIELD

Provided are methods and compositions, e.g., kits, for isolating protein or other biomolecules from biological or environmental samples containing humic substances, e.g. soil, compost, sediment, or manure samples.

### BACKGROUND

Protein sequences have a wide variety of applications in the field of molecular biology. They are a valuable tool in many analytical and application techniques used in the field of molecular biology, health and medicine, bioterrorism, forensics, space science, and food science. Some examples of these techniques include proteome mapping of microorganisms, detecting pathogens and beneficial microorganisms in soils, water, water filters, biofilms, plants and animals, and forensic identification of biological samples and environmental samples contaminated with different biological entities. All of these techniques are based on identifying a specific sequence of amino acids in either a biological sample, such as a microorganism, plant tissues or animal tissues, or in any environment capable of supporting life. Identifying target protein sequences directly in biological samples and in environmental samples has the advantages of speed, accuracy, high-throughput, and a low limit of detection of proteins. The target protein sequence, in order to be used as a diagnostic tool in such applications, should be free of contaminants that inhibit downstream applications. These contaminants are often from the groups that include phenolic and porphyrin substances, such as humic acids, fulvic acids, lignans, heme, chlorophyll, and quinones.

The field of protein extraction and subsequent analysis by mass spectrometry and other methods has enabled the rapid analysis of the composition of certain biological samples. However, the existing methods of protein extraction are often inadequate for isolation and purification of protein from environmental samples such as soil, manure, and plants that contain significant amounts of phenolic or porphyrin substances. In analysis of such samples, the protein is invariably co-extracted with the phenolic or porphyrin substances, thereby making it difficult to perform accurate analysis of the protein composition.

The nature of the contaminants in crude protein preparations from soils and sediments and their interactions with proteins are not well understood. Most frequently these contaminants are considered to be humic and fulvic acids and a heterogeneous mixture of phenolic polymers and oligomers. Humic substances are formed when microbes degrade plant residues and are stabilized to degradation by covalent binding of their reactive sites to metal ions and clay minerals. Humic substances consist of polycyclic aromatics to which saccharides, peptides, and phenols are attached. The predominant types of humic substances in soils are humic acids (molecular weight of 300 kDa and greater) and fulvic acids (molecular weight of as low as 0.1 kDa). Humic acids are soluble in alkaline pH and precipitate with hydrochloric or sulphuric acids at pH 1.0 to 2.0, while fulvic acids remain in solution even at acidic pH (Stevenson, 1994). Most frequently, protein extracts from soils showing brown coloration are indicative of contamination with humic-like substances. These brown compounds cannot be easily removed from protein extracts.

Standard methods for extraction of total protein involve thermally assisted detergent-based cellular lysis using, for example, SDS, followed by precipitation with trichloroacetic acid (TCA). An exemplary method is described by Chourey et al. (J. Proteome Res. 2010, 9(12):6615-22). Direct extraction of total protein from soils or sediments usually results in co-extraction of other soil components, mainly humic acids or other humic substances, which negatively interfere with protein detecting processes. Separation of humic substances from protein usually involves time-consuming and tedious steps. What is needed is a method for rapid isolation of protein from biological and environmental samples, in which the protein is effectively separated from the humic substances in the sample.

### SUMMARY

In one aspect, provided is a method for removing one or more contaminants from a biological or environmental sample, the method comprising the steps of: (a) contacting the sample with a first solution comprising a detergent, a buffer, one or more inorganic salts, and a polyol; and (b) contacting the resulting mixture of step (a) with a second solution comprising an inorganic salt. In some embodiments, the second solution comprises an inorganic salt which is sodium chloride. In some embodiments, the first solution further comprises a chelating agent. In some embodiments, the first solution further comprises an anti-foaming agent. In some embodiments, the first solution comprises two or more inorganic salts. In some embodiments, the method further comprises contacting the sample with a disulfide-reducing agent following step (a). In some variations the disulfide-reducing agent is DTT (dithiothreitol). In some embodiments, the method further comprises the step of agitating the resulting mixture of step (a), for example, in the presence of beads. In some embodiments, the method further comprises the step of agitating the resulting mixture of step (b), for example, in the presence of beads.

In another aspect, provided is a method for removing one or more contaminants from a biological or environmental sample, the method comprising the steps of: (a) contacting the sample with a first solution comprising a detergent, a buffer, one or more inorganic salts, and a polyol; (b) agitating the resulting mixture of step (a) in the presence of beads; (c) contacting the resulting mixture of step (b) with a second solution comprising an inorganic salt; and (d) agitating the resulting mixture of step (c) in the presence of beads. In some embodiments, the first solution further comprises a chelating agent. In some embodiments, the first solution further comprises an anti-foaming agent. In some embodiments, the second solution comprises an inorganic salt which is sodium chloride. In some embodiments, the method further comprises contacting the sample with a disulfide-reducing agent following step (a). In some variations, the disulfide-reducing agent is DTT.

In another aspect, provided is a method for purifying or isolating a biomolecule, e.g., protein, DNA, RNA, or lipid, from a sample comprising cells and one or more contaminants, the method comprising extracting the biomolecule into a solution, wherein the biomolecule is at least partially soluble in the solution, and the one or more contaminants are at least partially insoluble in the solution. In some embodiments, the method further comprises the step of lysing the cells. In some embodiments, the method further comprises the step of agitating the sample. In some embodiments, the method further comprises contacting the sample with a disulfide-reducing agent (e.g., DTT). In some embodiments, the biomolecule is partially soluble in the solution. In some embodiments, the biomolecule is mostly soluble in the solution. In some embodiments, the biomolecule is fully soluble in the solution. In some embodiments, the one or more contaminants are partially insoluble in the solution. In some embodiments, the one or more contaminants are mostly insoluble in the solution. In some embodiments, the one or more contaminants are fully insoluble in the solution.

In another aspect, provided is a method for purifying or isolating a biomolecule, e.g., protein, DNA, RNA, or lipid, from a sample comprising cells and one or more contaminants, the method comprising the steps of: (a) contacting the sample with a first solution comprising a detergent, a buffer, one or more inorganic salts, and a polyol; and (b) contacting the resulting mixture of step (a) with a second solution comprising an inorganic salt. In some embodiments, the first solution further comprises a chelating agent. In some embodiments, the first solution further comprises an anti-foaming agent. In some embodiments, the first solution comprises two or more inorganic salts. In some embodiments, the method further comprises contacting the sample with a disulfide-reducing agent (e.g., DTT) following step (a). In some embodiments, the method further comprises the step of agitating the resulting mixture of step (a) in the presence of beads. In some embodiments, the method further comprises the step of agitating the resulting mixture of step (b) in the presence of beads.

In another aspect, provided is a method for purifying or isolating a biomolecule, e.g., protein, DNA, RNA, or lipid, from a sample comprising cells and one or more contaminants, the method comprising the steps of: (a) contacting the sample with a first solution comprising a detergent, a buffer, one or more inorganic salts, and a polyol; (b) agitating the resulting mixture of step (a) in the presence of beads; and (c) contacting the resulting mixture of step (b) with a second solution comprising an inorganic salt; and (d) agitating the resulting mixture of step (c) in the presence of beads. In some embodiments, the first solution further comprises a chelating agent. In some embodiments, the first solution further comprises an anti-foaming agent. In some embodiments, the second solution comprises an inorganic salt which is sodium chloride. In some embodiments, the method further comprises contacting the sample with a disulfide-reducing agent (e.g., DTT) following step (a).

In another aspect, provided is a method for purifying or isolating a biomolecule, e.g., protein DNA, RNA, or lipid, from a sample comprising cells and one or more contaminants, the method comprising the steps of: (a) contacting the sample with a first solution comprising Tris Base, EDTA, KCl, MgCl₂, glycerol, and TRITON™ X-100 (4-(1,1,3,3-Tetramethylbutyl)phenyl-polyethylene glycol); and (b) contacting the resulting mixture of step (a) with a second solution comprising sodium chloride. In some embodiments, the first solution further comprises an anti-foaming agent.

In another aspect, provided is a method for purifying or isolating a biomolecule, e.g., protein DNA, RNA, or lipid, from a sample comprising cells and one or more contaminants, the method comprising the steps of: (a) contacting the sample with a first solution comprising Tris Base, EDTA, KCl, MgCl₂, glycerol, and Triton X-100; (b) agitating the resulting mixture of step (a) in the presence of beads; (c) contacting the resulting mixture of step (b) with a second solution comprising sodium chloride; and (d) agitating the resulting mixture of step (c) in the presence of beads. In some embodiments, the beads are glass or ceramic beads. In some embodiments, the first solution further comprises an anti-foaming agent.

In another aspect, provided is a kit comprising (a) a first solution comprising a detergent, a buffer, one or more inorganic salts, and a polyol; and (b) a second solution comprising an inorganic salt. In some embodiments, the first solution further comprises a chelating agent. In some embodiments, the first solution further comprises an anti-foaming agent. In some embodiments, the second solution comprises an inorganic salt which is sodium chloride. In some embodiments, the kit further comprises instructions describing a method for use according to any of the methods described herein. In some embodiments, the kit further comprises beads. In some embodiments, the kit further comprises an apparatus that can be used to agitate a sample in the presence of beads. In some embodiments, the kit further comprises an adaptor for connecting a vessel containing the sample to a vortex apparatus (e.g., Vortex Adapter, Mo Bio Laboratories, Carlsbad, Calif.). In one aspect, the kit comprises one or more tube vessels useful for performing the method of use. Where tube vessels are included in the kit, the vessels can be sterile. In some embodiments, the kit includes components useful for further processing an isolated biomolecule, e.g., protein, DNA, RNA, or lipid.

In another aspect, provided is a kit comprising (a) a first solution comprising Tris Base, EDTA, KCl, MgCl₂, glycerol, and TRITON™ X-100 (4-(1,1,3,3-Tetramethylbutyl)phenyl-polyethylene glycol), (b) a second solution comprising sodium chloride, and (c) glass or ceramic beads. In some embodiments, the kit further comprises instructions describing a method of use according to any of the methods described herein. In some embodiments, the first solution further comprises an anti-foaming agent.

### BRIEF DESCRIPTION OF THE DRAWINGS

FIG. 1 shows an SDS-PAGE gel of E. coli-spiked sterile soils prepared according to the procedure described in Example 1.

FIG. 2 shows protein extracted from 5 g of E. coli-spiked soil using thermally assisted detergent-based cellular lysis with SDS, followed by TCA precipitation.

FIG. 3 shows crude protein extracts prior to protein precipitation. The extract on the left was prepared using the procedure described in Example 1. The extract on the right was prepared using the procedure described by Chourey et al. (supra). The darker color of the extract on the right is a result of co-extraction of humic substances. Proteins were extracted from 5 g of an organically rich soil.

### DETAILED DESCRIPTION

Provided are methods and compositions for detecting and/or isolating a biomolecule, e.g., protein, DNA, RNA, or lipid, and/or for detecting an organism, e.g., a microorganism, in a sample, e.g., a biological or environmental sample. Provided are methods and compositions, e.g., kits, for isolating a biomolecule from sources containing contaminating substances that interfere with use of the purified biomolecules in subsequent applications. In one aspect, provided are methods and kits for purifying a biomolecule, e.g., protein, DNA, RNA, or lipid, from a biological or environmental sample to be free of contaminants that may impede analysis or identification of the biomolecules. The environmental samples include but are not limited to soil, sediment, sludge, decomposing biological matter, archaeological remains, peat bogs, compost and water that are terrestrial or subterranean in origin. Biomolecules isolated using the kits and methods provided herein may be used in the areas of molecular biological application, including, for example, analytical, cloning, diagnostic, and detection in the fields of agriculture, horticulture, forestry, forensics, biological research, organism and sample composition identification, characterization, applied microbiology, proteomics, environmental analysis, water testing, and combating bioterrorism.

As used herein, unless clearly indicated otherwise, the terms “a,” “an,” “the,” and the like refer to one or more.

As used herein, the terms “including,” “containing,” and “comprising” are used in their open, non-limiting sense.

To provide a more concise description, some of the quantitative expressions given herein are not qualified with the term “about.” It is understood that, whether the term “about” is used explicitly or not, every quantity given herein is meant to refer to that actual given value, and it is also meant to refer to the approximation to such given value that would reasonably be inferred based on the ordinary skill in the art, including equivalents and approximations due to the experimental and/or measurement conditions for such given value.

The term “biological sample” as used herein, refers to a sample obtained from a biological subject, including sample of biological tissue or fluid origin obtained in vivo or in vitro. Such samples can be, but are not limited to, body fluid (e.g., blood, blood plasma, serum, or urine), organs, tissues, stool, swab samples, fractions and cells isolated from mammals (e.g., humans), biofilms (e.g., oral biofilms, environmental biofilms), filtered water samples. Biological samples also may include sections of the biological sample including tissues (e.g., sectional portions of an organ or tissue). The term “biological sample” may also include extracts from a biological sample, for example, an antigen from a biological fluid (e.g., blood or urine). A biological sample may be of prokaryotic origin (e.g., bacteria, archaea) or eukaryotic origin (e.g., fungi, plants, insects, protozoa, birds, fish, reptiles). In some embodiments, the biological sample is mammalian (e.g., rat, mouse, cow, dog, donkey, guinea pig, or rabbit). In certain embodiments, the biological sample is of primate origin (e.g., example, chimpanzee or human).

The terms “environmental” and “environmental sample”, include any environmental material, e.g., material contained in the earth and space, including space dust, airborne and waterborne locations and will include any organism, structure, and component considered alive, dead, dormant or inactive, whole, complete, undecaying and decaying that contains a biomolecule, e.g., protein, DNA, RNA, or lipid. “Environmental” and “environmental sample” include material and organisms that may be isolated from the environment as dust or suspended material collected by filtration.

The term “soil” as used herein refers to environmental samples of soil, sediment, manure, compost, and the like, e.g., commercial potting mixtures, commercial soil amendments. The term also includes a broad range of organic carbon and nitrogen content and varying sand, silt and/or clay compositions. “Soil” includes any composition containing components commonly associated with habitable and uninhabitable areas of the earth and space, including for example varying descriptions, e.g., indoor dust, outdoor dust, dirt, mud, muck, silt, ground, sewage, compost, composting landfills at various depths. Examples of soil samples include but are not limited to landfill (e.g., 0-3 inches deep or 3-6 inches deep); late-stage compost; coffee compost; marine sediment; lake sediment; mud sediment; animal manure (e.g., horse manure); mulch, e.g., mulch top soil; the ocean floor, hillsides, mountaintops and may extend from the surface to any depth. The sample may be collected by any means using any commercially available or improvised method and tested directly. A biomolecule, e.g., protein, DNA, RNA, or lipid, may be extracted using a kit or method provided herein at the site of collection, or the sample may be stored before a biomolecule, e.g., protein, DNA, RNA, or lipid, is isolated therefrom.

### Methods and Compositions

In some aspects of any of the methods and compositions described herein, the one or more contaminants include, without limitation, humic substances, such as humic acids, fulvic acids, and lignans, heme, chlorophyll, and quinones. In some embodiments, contaminants include phenolic or porphyrin compounds other than proteins, oligopeptides, amino acids, DNA, RNA, oligonucleotides, nucleic acids, and lipids. In some embodiments, contaminants are phenolic or porphyrin components of natural organic matter in soil and water as well as in geological organic deposits such as lake sediments, peats, brown coals, and shales. In some embodiments, contaminants are complex and heterogeneous mixtures of polydispersed materials formed by reactions during the decay and transformation of plant and microbial remains and may be derived from components such as plant lignin, polysaccharides, melanin, cutin, proteins, lipids, nucleic acids, and fine char particles.

In some aspects of any of the methods and compositions described herein, the first solution is added to the sample prior to addition of the second solution. The first solution may be added to the sample at room temperature, or it may be added at a temperature below room temperature. The sample may be kept on ice or otherwise kept below room temperature before, during, or after addition of the first solution. In some instances, a disulfide-reducing agent (e.g., dithiothreitol) is added to the solution following addition of the first solution and prior to addition of the second solution. Addition of the first solution, optionally followed by addition of a disulfide-reducing agent, may create a hypotonic environment for the sample.

In some variations, the sample is agitated by any of the methods described herein after addition of the first solution or after addition of the disulfide-reducing agent. Agitation may be for 1, 2, 5, 10, 15, 20, 25, 30, 40, 50, or 60 minutes. Agitation may be carried out at room temperature or at a temperature below room temperature, for example, at 4° C. Agitation at low temperature may be desirable for native protein extraction. In some variations, the sample may be incubated on at room temperature or at a temperature below room temperature, for example, at 4° C., prior to or following agitation. Incubation may be for 1, 2, 5, 10, 15, 20, 25, 30, 40, 50, or 60 minutes.

In some variations, the sample is centrifuged prior to addition of the second solution. Centrifugation may help remove residual soil, beads, buffer, or other components of the sample solution from the sides or cap of the vessel containing the solution before adding the second solution. The sample may be centrifuged at room temperature or at a temperature below room temperature, for example, at 4° C. The sample may be centrifuged in a refrigerated centrifuge.

In some variations, the sample is agitated by any of the methods described herein after addition of the second solution or after addition of the disulfide-reducing agent. Agitation may be for 1, 2, 5, 10, 15, 20, 25, 30, 40, 50, or 60 minutes. Agitation may be carried out at room temperature or at a temperature below room temperature, for example, at 4° C. Agitation at low temperature may be desirable for native protein extraction. In some variations, the sample may be incubated on at room temperature or at a temperature below room temperature, for example, at 4° C., prior to or following agitation. Incubation may be for 1, 2, 5, 10, 15, 20, 25, 30, 40, 50, or 60 minutes.

In some variations, the sample is centrifuged following addition of the second solution. Centrifugation may help separate the extracted biomolecule, e.g., protein, DNA, RNA, or lipid, from the soil particles, bead, and other undissolved material in the vessel. The sample may be centrifuged at room temperature or at a temperature below room temperature, for example, at 4° C. The sample may be centrifuged in a refrigerated centrifuge. Following centrifugation, the supernatant may be collected and centrifuged an additional time. This may help to remove fine soil particles and other undissolved material from the extracted biomolecule, e.g., protein, DNA, RNA, or lipid.

In some aspects of any of the methods and compositions described herein, the first solution comprises a detergent, a buffer, one or more inorganic salts, and a polyol. In some variations, the first solution further comprises a chelating agent. In some variations, the first solution further comprises an anti-foaming agent.

In some embodiments, the first solution contains a detergent. The detergent may be any non-ionic detergent. Particular detergents that can be used in the compositions and methods described herein include, without limitation, TRITON™ X-100 (4-(1,1,3,3-Tetramethylbutyl)phenyl-polyethylene glycol), TRITON™ X-114, BRIJ® 58 (polyethylene glycol hexadecyl ether), BRIJ® 35 (polyethylene glycol dodecyl ether), sodium taurocholate, TWEEN™ 20, TWEEN™ 80, polysorbate 20, polysorbate 80, NP-40 (nonyl phenoxypolyethoxylethanol), CHAPS 3-[(3-cholamidopropyl)dimethylammonio]-1-propanesulfonate), and CHAPSO (3-[(3-cholamidopropyl)dimethylammonio]-2-hydroxy-1-propanesulfonate). The first solution may contain 0.01 to 2, 0.1 to 1, 0.5 to 1, 0.5 to 1.5 or 0.75 to 1.25 vol % detergent. The first solution may contain at least about 0.01, about 0.1, about 0.5, about 0.75, or about 1 vol % detergent. The first solution may contain up to about 0.1, about 0.25, about 0.5, about 0.75, about 1, about 1.5, or about 2 vol % detergent. In some instances, the first solution contains 0.1 to 1 vol % TRITON™ X-100(4-(1,1,3,3-Tetramethylbutyl)phenyl-polyethylene glycol) detergent. In other instances, the first solution contains about 1 vol % Triton X-100 detergent.

In some embodiments, the first solution contains a chelating agent. The chelating agent may be EDTA (ethylenediaminetetraacetic acid), or it may be a salt form of EDTA, such as a sodium, potassium, or calcium salt of EDTA. The first solution may contain 0.01 to 4, 0.1 to 3, 0.1 to 2, 1 to 2, 1.5 to 2.5, 0.1 to 1, 0.5 to 2, or 0.5 to 1 mM chelating agent. The first solution may contain at least about 0.01, about 0.1, about 0.25, about 0.5, about 0.75, about 1, about 1.25, about 1.5, about 2, or about 3 mM chelating agent. The first solution may contain up to about 0.1, about 0.25, about 0.5, about 0.75, about 1, about 1.25, about 1.5, about 2, about 3, or about 4 mM chelating agent. In some instances, the first solution contains 0.1 to 2 mM EDTA. In other instances, the first solution contains about 2 mM EDTA.

In some embodiments, the first solution contains a buffer. The buffer may be a basic buffer. The buffer may be any buffer that is amenable to culturing cells. The buffer may be any buffer that is useful for maintaining the first solution at physiological pH. Particular buffers that can be used in the compositions and methods described herein include, without limitation, Tris Base (tris(hydroxymethyl)aminomethane), Bis-Tris (Bis(2-hydroxyethyl)-amino-tris(hydroxymethyl)-methane), HEPES (4-(2-hydroxyethyl)-1-piperazineethanesulfonic acid), PBS (phosphate buffered saline), MOPS (3-(N-morpholino)propanesulfonic acid), MES (2-(N-morpholino)ethanesulfonic acid), and CAPS (N-cyclohexyl-3-aminopropanesulfonic acid). The first solution may contain 1 to 200, 10 to 100, 10 to 80, 1 to 50, 1 to 20, 10 to 50, 10 to 30, 10 to 20, 20 to 50, or 15 to 25 mM buffer. The first solution may contain at least about 1, about 10, about 20, about 30, about 40, about 50, about 60, about 70, about 80, about 90, or about 100 mM buffer. The first solution may contain up to about 10, about 20, about 30, about 40, about 50, about 60, about 70, about 80, about 90, about 100, about 150, or about 200 mM buffer. In some instances, the first solution contains 10 to 100 mM Tris Base buffer. In other instances, the first solution contains about 20 mM Tris Base buffer.

In some embodiments, the first solution contains a polyol. In some variations, the polyol is a disaccharide. Particular polyols that can be used in the compositions and methods described herein include, without limitation, glycerol, sucrose, and trehalose. In a particular variation, the polyol is glycerol. The first solution may contain 1 to 30, 5 to 20, 5 to 10, 10 to 20, 5 to 15, or 10 to 15 vol % polyol. The first solution may contain at least about 1, about 5, about 10, about 15, about 20, or about 25 vol % polyol. The first solution may contain up to about 5, about 10, about 15, about 20, about 25, or about 30 vol % polyol. In some instances, the first solution contains 5 to 20 vol % glycerol. In other instances, the first solution contains 10 vol % glycerol.

In some embodiment, the first solution contains one or more salts. The salts may be inorganic salts, such as sodium salts, potassium salts, calcium salts, magnesium salts, chloride salts, bicarbonate salts, or sulfate salts. In some variations, the salts contain monovalent cations or divalent cations. Particular salts that can be used in the compositions and methods described herein include, without limitation, KCl, NaCl, NaHCO₃, NaSO₄, MgCl₂, and CaCl₂. In some embodiments, the first solution contains two or more different salts, such as a potassium salt and a magnesium salt. In some embodiments, the first solution contains one salt selected from MgCl₂ and CaCl₂, and one salt selected from KCl, NaCl, NaHCO₃, and NaSO₄. In some instances, the first solution contains MgCl₂ and KCl. In some variations, the first solution contains CaCl₂ and does not contain NaCl. The first solution may contain 1 to 300, 10 to 250, 20 to 200, 10 to 100, 20 to 50, 10 to 30, or 20 to 40 mM salt. The first solution may contain at least about 1, about 10, about 20, about 30, about 40, about 50, about 60, about 70, about 80, about 90, about 100, about 150, about 200, or about 250 mM salt. The first solution may contain up to about 10, about 20, about 30, about 40, about 50, about 60, about 70, about 80, about 90, about 100, about 150, about 200, about 250, or about 300 mM salt. In some instances, the first solution contains 10 to 100 mM salt selected from KCl, NaCl, NaHCO₃, and NaSO₄, and 10 to 100 mM salt selected from MgCl₂ and CaCl₂. In some instances, the first solution contains 10 to 100 mM KCl and 10 to 100 mM MgCl₂. In other instances, the first solution contains about 10 mM KCl and about 10 mM MgCl₂.

In some embodiments, the first solution contains an anti-foaming agent. The anti-foaming agent may be a silica-based anti-foaming agent. Particular anti-foaming agents that can be used in the compositions and methods described herein include 100% active silicone, poly(methylsiloxane) in silicone oil, or silicone emulsions. Particular silicone emulsions contain from 10 to 30% silicone and one or more emulsifiers.

In some variations, the first solution contains Tris Base, KCl, MgCl₂, glycerol, TRITON™ X-100 (4-(1,1,3,3-Tetramethylbutyl)phenyl-polyethylene glycol), and EDTA. In a particular variation, the first solution contains about 20 mM Tris Base, about 2 mM EDTA, about 10 mM KCl, about 10 mM MgCl₂, about 10% glycerol, and about 1% TRITON™ X-100 (4-(1,1,3,3-Tetramethylbutyl)phenyl-polyethylene glycol).

In some variations, the second solution contains one or more salts. The salts may be inorganic salts, such as sodium salts, potassium salts, calcium salts, magnesium salts, chloride salts, bicarbonate salts, or sulfate salts. In some variations, the salts contain monovalent cations or divalent cations. In some variations, the salts contain monovalent cations. Particular salts that can be used in the compositions and methods described herein include, without limitation, KCl, NaCl, NaHCO₃, NaSO₄, MgCl₂, and CaCl₂. In some embodiments, the first solution contains a sodium salt or a potassium salt. In some embodiments, the first solution contains a chloride salt. In some embodiments, the second solution contains NaCl. In some embodiments, the second solution contains MgCl₂. In some embodiment, the second solution contains CaCl₂. The second solution may contain 1 to 6, 2 to 5, 3 to 5, 4 to 5, 3.5 to 4.5, or 4 to 4.5 M salt. The second solution may contain at least about 1, about 2, about 3, about 4, or about 5 M salt. The second solution may contain up to about 1, about 2, about 3, about 4, about 5 or about 6 M salt. In some variations, the total concentration of salt in the sample after addition of the second solution is 0.01 to 1, 0.1 to 0.5, 0.2 to 0.4, 0.1 to 0.3, or 0.3 to 0.5 M. In some variations, the total concentration of salt in the sample after addition of the second solution is at least about 0.01, about 0.1, about 0.2, about 0.3, about 0.4, about 0.5, about 0.6, or about 0.7 M. In some variations, the total concentration of salt in the sample after addition of the second solution is up to about 0.1, about 0.2, about 0.3, about 0.4, about 0.5, about 0.6, about 0.7 M, or about 1 M. In some variations, the second solution contains 4.2 M NaCl. In some variations, the total concentration of NaCl in the sample after addition of the second solution is about 140 to 500 mM.

The methods and compositions described herein may comprise one or more steps or components for agitation of the sample. Agitation may be achieved by any method known in the art, including, without limitation, sonication, blending, mechanical homogenization (e.g., shear homogenization, rotor-stator homogenization), manual homogenization (e.g., mortar and pestle or dounce homogenization), or high pressure homogenization (e.g., French Pressure Cell). Agitation may be carried out at room temperature or at a temperature below room temperature. In some embodiments, the sample is agitated at about 4° C. In some embodiments, the sample is agitated in the presence of beads (i.e. “bead beating”). The beads may be homogenizing beads. The beads may be made of any solid material that is non-reactive with the samples, solutions, or other reagents used in the method. The beads may be round or irregularly shaped. The beads may be of uniform size or of varying sizes. The beads may be of uniform material or of heterogeneous material. In some variations, the beads are ceramic. In some variations, the beads are glass. In some variations, the beads have an average diameter of 0.01 to 10, 0.1 to 5, 0.1 to 3, 0.2 to 3, 0.1 to 2, or 1 to 3 mm. In some variation the beads have an average diameter of at least about 0.01, about 0.1, about 0.2, about 0.5, about 1, about 2, about 3, or about 5 mm. In some variations, the beads have an average diameter of up to about 0.1, about 0.2, about 0.5, about 1, about 2, about 3, about 5, or about 10 mm. In some instances, the beads are 0.2, 1.4, or 2.8 mm ceramic beads. In some instances, the beads are 0.1 or 0.5 mm glass beads. In a particular variation, the beads are 0.2 mm ceramic beads. In another particular variation, the beads are 0.1 mm glass beads. In yet another particular variation, the beads are a mixture of 0.1 mm glass beads and 0.2 mm ceramic beads. Agitation of the sample in the presence of beads may be achieved by physical force, such as shaking or vibration. Vibration can be introduced by any convenient means, such as by a sonication or a vortex apparatus using a Vortex Adapter (Mo Bio Laboratories, Carlsbad, Calif.), for example.

The methods and compositions described herein can be used to remove one or more contaminants from a sample that contains a biomolecule, e.g., protein, DNA, RNA, or lipid. The resulting a biomolecule, e.g., protein, DNA, RNA, or lipid, may contain less than 40%, less than 30%, less than 20%, less than 10%, less than 5%, less than 2%, less than 1%, less than 0.1%, less than 0.01%, less than 0.001%, or less than 0.0001% by weight of contaminants. In some instances, the purity of the resulting protein or other biomolecule is at least 50%, at least 60%, at least 70%, at least 80%, at least 90%, at least 95%, at least 98%, at least 99%, at least 99.9%, or at least 99.99% by weight. In some instances, at least 50%, at least 60%, at least 70%, at least 80%, at least 90%, at least 95%, at least 98%, at least 99%, at least 99.9%, or at least 99.99% by weight of the contaminants present in the sample are removed using any of the methods or compositions described herein.

Any of the methods and compositions described herein can be applied to a biological or environmental sample of any scale. In some variations, the sample is a 10, 50, 100, or 500 mg sample. In some variations, the sample is a 1, 2, 5, 10, 20, 50, 100, 500, or 1,000 gram sample.

The methods and compositions described herein may be used to recover a biological molecule from a biological or environmental sample. The biological molecule may be a protein, DNA, RNA, or lipid. In some variations, the protein recovered from the sample is denatured protein. Addition of DTT to the sample (e.g., to a final concentration of 10 mM) following addition of the first solution may facilitate recovery of denatured protein. Agitation of the sample in the presence of beads following addition of the first solution to the sample and/or following addition of the second solution to the sample may also facilitate recovery of denatured protein. In other variations, the protein recovered from the sample is native protein or extracellular protein. Addition of DTT to the sample (e.g., to a final concentration of 1 mM) and/or addition of protease inhibitors following addition of the first solution may facilitate recovery of native or extracellular protein. For recovery of native or extracellular protein, agitation of the sample in the presence of beads may be for a shortened time period, or the agitation step may be absent.

Any of the methods described herein may also include subsequent steps to further separate, isolate, or purify a biomolecule, e.g., protein, DNA, RNA, or lipid, from the contaminants. For example, the solution containing a dissolved biomolecule, e.g., protein, DNA, RNA, or lipid, can be substantially removed from contact with the undissolved portions of the sample and any other components with which it is in contact, such as homogenizing beads, via methods known in the art. Such methods include filtration (e.g., microfiltration, ultrafiltration) and centrifugation (e.g., ultracentrifugation). In some instances, multiple centrifugation and/or filtration steps may be used. Centrifugation or filtration may be carried out at a temperature below room temperature (e.g., 4° C.) to minimize degradation of the biomolecule, e.g., protein, DNA, RNA, or lipid.

After filtration or centrifugation, the biomolecule, e.g., protein, DNA, RNA, or lipid, may be precipitated out of the solution or supernatant by addition of a precipitation agent, such as trichloroacetic acid (TCA). For complete precipitation to occur, incubation for 10 minutes to 24 hours may be required. Subsequent washing with acetone or other organic solvent may be performed to remove residual TCA and/or detergent. The sample may be pelleted using centrifugation. The washing and pelleting steps may be repeated multiple times.

### Applications

The methods and compositions described herein have many medical and veterinary applications, e.g., for diagnosis, prognosis, epidemiology, inspection of contamination of materials (e.g., drugs, dressing, instruments, implants), foods (e.g., inspections of meat, vegetables, seafood, etc.), including medical and veterinary analysis of feces (including manure analysis for animals). Medical and veterinary applications include detection of soils, e.g., for bioterrorism purposes, e.g., anthrax, viruses, nematodes, and the like. Virus detection using the compositions and methods provided herein can also be used to analyze manure and soil, water, water filters, biofilms, air and the like. Viruses that can be detected by compositions and methods provided herein include enterovirus, norovirus, variola, varicella, reovirus, retroviruses (e.g., HIV), viral hemorrhagic fevers (e.g., Ebola, Marburg, Machupo, Lassa), Variola major, viral encephalitis and the like, as listed in Table 1, below. The compositions and methods provided herein can also be used to detect spores, toxins and biologically produced poisons, for example, by detecting Bacillus anthracis, anthrax spores are also detected (albeit, indirectly), detection of Clostridium perferinges implies presence of toxin, etc.

Examples of Gram negative bacteria that can be detected and/or whose biomolecules, e.g. proteins, DNA, RNA, or lipids, can be isolated using the kits and methods provided herein include but are not limited to Gram negative rods (e.g., anaerobes such as bacteroidaceae (e.g., Bacteroides fragilis), facultative anaerobes, enterobacteriaceae (e.g., Escherichia coli), vibrionaceae (e.g., Vibrio cholerae), pasteurellae (e.g., Haemophilus influenzae), and aerobes such as pseudomonadaceae (e.g., Pseudomonas aeruginosa); Gram negative cocci (e.g., aerobes such as Neisseriaceae (e.g., Neisseria meningitidis) and Gram negative obligate intracellular parasites (e.g., Rickettsiae (e.g., Rickettsia spp.). Examples of Gram negative bacteria families that can be detected and/or whose biomolecules, e.g. proteins, DNA, RNA, or lipids, can be isolated include but are not limited to Acetobacteriaceae, Alcaligenaceae, Bacteroidaceae, Chromatiaceae, Enterobacteriaceae, Legionellaceae, Neisseriaceae, Nitrobacteriaceae, Pseudomonadaceae, Rhizobiaceae, Rickettsiaceae and Spirochaetaceae.

Examples of Gram positive bacteria that can be detected and/or whose biomolecules, e.g. proteins, DNA, RNA, or lipids, can be isolated using the kits and methods provided herein include but are not limited to A. globiformis, B. subtilis, C. renale, M. luteus, R. erythropolis, Ea39, Ben-28 and S. lividans. Gram positive bacteria that can be detected and/or whose biomolecules, e.g. proteins, DNA, RNA, or lipids, can be isolated also are in groups that include, for example, Corynebacterium, Mycobacterium, Nocardia; Peptococcus (e.g., P. niger); Peptostreptococcus (e.g., Ps. anaerobius; some species in the group form clumps and clusters; some species in the group form diplococci (the latter of which are distinguished by their ability to form butyrate); and some species in the group are capable of fermentation, reduction of nitrate, production of indole, urease, coagulase or catalase); Ruminococcus; Sarcina; Coprococcus; Arthrobacter (e.g., A. globiformis, A. citreus or A. nicotianae); Micrococcus (e.g., M. luteus (previously known as M. lysodeikticus), M. lylae, M. roseus, M. agilis, M. kristinae and M. halobius); Bacillus (e.g., B. anthracis, B. azotoformans, B. cereus, B. coagulans, B. israelensis, B. larvae, B. mycoides, B. polymyxa, B. pumilis, B. stearothormophillus, B. subtilis, B. thuringiensis, B. validus, B. weihenstephanensis and B. pseudomycoides); Sporolactobacillus; Sporocarcina; Filibacter; Caryophanum and Desulfotomaculum. Other Gram positive bacteria that can be detected and/or whose biomolecules, e.g. proteins, DNA, RNA, or lipids, can be isolated fall into the group Clostridium, which often include peritrichous flagellation, often degrade organic materials to acids, alcohols, CO₂, H₂ and minerals (acids, particularly butyric acid, are frequent products of clostridial fermentation), and in one aspect form ellipsoidal or spherical endospores, which may or may not swell the sporangium. Species of Clostridium that can be detected and/or whose nucleic acid can be isolated include psychrophilic, mesophilic or thermophilic species, saccharolytic species, proteolytic species and/or specialist species, and those that are both saccharolytic and proteolytic species. Saccharolytic species of Clostridium that can be detected and/or whose biomolecules, e.g. proteins, DNA, RNA, or lipids, can be isolated include but are not limited to Cl. aerotolerans, Cl. aurantibutyricum, Cl. beijerinckii, Cl. botulinum B,E,F*, Cl. butyricum, Cl. chauvoei, Cl. difficile, Cl. intestinale, Cl. novyi A, Cl. pateurianum, Cl. saccharolyticum, Cl. septicum, Cl. thermoaceticum, and Cl. thermosaccharolyticum.

Proteolytic species of Clostridium that can be detected and/or whose biomolecules, e.g. proteins, DNA, RNA, or lipids, can be isolated include but are not limited to Cl. argeninense, Cl. ghoni, Cl. limosum, Cl. putrefaciens, Cl. subterminale and Cl. tetani. Species that are proteolytic and saccharolytic that can be detected and/or whose biomolecules, e.g. proteins, DNA, RNA, or lipids, can be isolated include but are not limited to Cl. acetobutylicum, Cl. bifermenans, Cl. botulinum A, B, F (prot.) *, Cl. botulinum C,D*, Cl. cadaveris, Cl. haemolyticum, Cl. novyi B,C,* Cl. perfringens, Cl. putrefaciens, Cl. sordelli and Cl. sporogenes. As indicated by an asterisk, Cl. botulinum is subdivided into a number of types according to the serological specificities of the toxins produced. Specialist Clostridium species that can be detected and/or whose biomolecules, e.g. proteins, DNA, RNA, or lipids, can be isolated include but are not limited to Cl. acidiurici, Cl. irregularis, Cl. kluyveri, Cl. oxalicum, Cl. propionicum, Cl. sticklandii and Cl. villosum. These specificities are based on neutralization studies. Other Clostridium species that can be detected and/or whose biomolecules, e.g. proteins, DNA, RNA, or lipids, can be isolated include those that produce botulinum toxins.

Examples of fungi that can be detected and/or whose biomolecules, e.g. proteins, DNA, RNA, or lipids, can be isolated using the kits and methods provided herein include but are not limited to Halocyphina villosa, Hypoxylon oceanicum, Verruculina enalia, Nia vibrissa, Antennospora quadricornuta, Lulworthia spp. and Aigialus parvus. Examples of algae that can be detected and/or whose biomolecules, e.g. proteins, DNA, RNA, or lipids, can be isolated include but are not limited to brown algae (e.g., Phylum Phaeophycota Dictyota sp. (Class Phaeophyceae, Family Dictyotaceae); green algae (e.g., Phylum Chlorophycota Chaetomorpha gracilis (Class Chlorophyceae, Family Cladophoraceae); and red algae (e.g., Phylum Rhodophycota, Catenella sp. (Class Rhodophyceae, Family Rhabdoniaceae).

Organisms that can be detected by the kits and methods provided herein in a sample, e.g., an agricultural soil, include but are not limited to Pseudomonas spp., Serratia spp., Bacillus spp., Flavobacterium spp., Actinomycetes and fungi; in polluted soils include but are not limited to Pseudomonas spp. and Xanthomonas spp.; in marsh/sediments include but are not limited to Escherichia spp., Proteus spp., Methanogens and Actinomycetes; and in forest soils include but are not limited to Mycorrhizae, Fungi and Actinomycetes. An example of a bacterium detected in soil samples for use in combating bioterrorism using methods and kits provided herein is Bacillus anthracia.

Pathogens and toxins that can be detected by kits and methods provided herein include, without limitation, those listed in Table 1, below:

TABLE 1

| | | CDC | Specific | General | Detection | 1° |
|---|---|---|---|---|---|---|
| Disease/Type | Organism/agent | Group | Type | Class | Type | Target |
| Anthrax | Bacillus anthracis | A | G+ Spore | Bacterium | DNA | Human |
| Plague | Yersinia pestis | A | G− Veg | Bacterium | DNA | Human |
| Tularemia | Francisella | A | G− Veg | Bacterium | DNA | Human |
| | tularensis | | | | | |
| Brucellosis | Brucella spp. | B | G− Veg | Bacterium | DNA | Human |
| Glanders | Burkholderia mallei | B | G− Veg | Bacterium | DNA | Human |
| Melioidosis | Burkholderia | B | G− Veg | Bacterium | DNA | Human |
| | pseudomallei | | | | | |
| Psittacosis | Chlamydia psittaci | B | G− Veg | Bacterium | DNA | Human |
| Q Fever | Coxiella burnettii | B | Gv Veg | Bacterium | DNA | Human |
| Typhus fever | Rickettsia | B | Gv Veg | Bacterium | DNA | Human |
| | prowazekii | | | | | |
| Smallpox | Variola major | A | Virus | Virus | DNA | Human |
| Viral | Ebola | A | Filovirus | Virus | RNA | Human |
| hemorrhagic | | | | | | |
| fevers | | | | | | |
| Viral | Marburg | A | Filovirus | Virus | RNA | Human |
| hemorrhagic | | | | | | |
| fevers | | | | | | |
| Viral | Machupo | A | Arenavirus | Virus | RNA | Human |
| hemorrhagic | | | | | | |
| fevers | | | | | | |
| Viral | Lassa | A | Arenavirus | Virus | RNA | Human |
| hemorrhagic | | | | | | |
| fevers | | | | | | |
| Viral | Venezuelan Equine | B | Alphavirus | Virus | RNA | Human |
| encephalitis | Encephalitis | | | | | |
| Viral | Eastern Equine | B | Alphavirus | Virus | RNA | Human |
| encephalitis | Encephalitis | | | | | |
| Viral | Western Equine | B | Alphavirus | Virus | RNA | Human |
| encephalitis | Encephalitis | | | | | |
| Viral infection | Echovirus | N/A | Enterovirus | Virus | RNA/ | Human |
| | | | | | Protein | |
| Poliomyelitis | Poliovirus | N/A | Enterovirus | Virus | RNA/ | Human |
| | | | | | Protein | |
| Common cold | Rhinovirus | N/A | Enterovirus | Virus | RNA/ | Human |
| | | | | | Protein | |
| Hand, food, | Coxsackie A virus | N/A | Enterovirus | Virus | RNA/ | Human |
| and mouth | | | | | Protein | |
| disease | | | | | | |
| Viral infection | Coxsackie B virus | N/A | Enterovirus | Virus | RNA/ | Human |
| | | | | | Protein | |
| Viral infection | Other Enteroviruses | N/A | Enterovirus | Virus | RNA/ | Human |
| | | | | | Protein | |
| Viral | Norovirus | N/A | Calicivirus | Virus | RNA/ | Human |
| gastroenteritis | | | | | Protein | |
| Hepatitis A | Hepatitis A virus | N/A | Picomavirus | Virus | RNA/ | Human |
| | | | | | Protein | |
| Hepatitis B | Hepatitis B virus | N/A | Hepadnavirus | Virus | DNA/ | Human |
| | | | | | Protein | |
| Hepatitis C | Hepatitis C virus | N/A | Togavirus | Virus | RNA/ | Human |
| | | | | | Protein | |
| Botulism | Clostridium | A | Toxin | Toxin | Protein | Human |
| | botulinum toxin | | | | | |
| Toxins | Ricinus communis | B | Toxin | Toxin | Protein | Human |
| Toxins | Staph. aureus | B | Enterotoxin | Toxin | Protein | Human |
| | | | B | | | |
| Toxins | Clostridium | B | Epsilon | Toxin | Protein | Human |
| | perferinges toxin | | Toxin | | | |

The kits and methods provided herein can be used to isolate the total protein in a biological or environmental sample, or they may be used to isolate one or more specific proteins in the sample, for example, in order to assess the activity of such specific protein. The specific protein may be, for example, a fungal protein or a protein from a Gram positive bacterium. The specific protein may be an extracellular protein. It may be desirable, in some instances, to collect native (e.g., undigested) protein from the sample. In other instances, it may be desirable to collect digested protein.

Biomolecules, e.g., protein, DNA, RNA, or lipids, isolated or purified using any of the methods or compositions described herein can be detected, analyzed, characterized, and/or further purified using any method known in the art. Particular methods of detecting, analyzing, characterizing, and/or further purifying biomolecules, e.g., protein, DNA, RNA, or lipids, isolated or purified using any of the methods or compositions described herein include 1-dimensional polyacrylamide gel electrophoresis (1D PAGE), 2-dimensional polyacrylamide gel electrophoresis (2D PAGE), ELISA-type assays for assessment of native protein activity, other enzyme-based assays, western blotting, sequencing, and antibody production (e.g., injecting proteins into animals such as rabbits or making monoclonal antibodies).

The compositions and methods will be further described with reference to the following examples; however, it is to be understood that the kits and methods are not limited to such examples.

### EXAMPLES

### Example 1

### Protein Isolation from an Environmental Sample

Sterile soil samples from various sources (lake sediment, lagoon sediment, beach sand, and agricultural soil) were spiked with E. coli. Five grams of a particular E. coli-spiked soil sample were added to 50 ml bead tubes containing a mixture of 0.1 mm glass and 0.2 mm ceramic beads and placed on ice. The first solution was added to the soil (15 ml) followed by addition of dithiothreitol (DTT) to a final concentration of 10 mM. Samples were vortexed to mix and incubated on ice for 10 minutes at 4° C. Following ice incubation, sample tubes were vortexed for 10 minutes with the vortex set to the highest speed. A MO BIO Vortex Adapter for 50 ml tubes was utilized for the bead beating step. Samples were collected in the tube by brief centrifugation in a refrigerated centrifuge at 4° C. The second solution was added (1.5 ml) and samples vortexed to mix and incubated on ice at 4° C. for 30 minutes. This was followed by a second round of bead beating on the vortex in the 50 ml tube adapters for 10 minutes at the highest setting. Samples were collected by centrifugation at 4500×g, in a refrigerated centrifuge set at 4° C. for 20 minutes.

The supernatant containing the protein was transferred to a clean 50 ml centrifuge tube and centrifuged again at 4500×g in refrigerated centrifuge set at 4° C. for 20 minutes. The supernatant was transferred to a clean 50 ml centrifuge tube. Samples were precipitated using 0.25 ml of 100% trichloroacetic acid (TCA) to each 1 ml of supernatant, vortexed to mix, and incubated overnight at −20° C.

The precipitated protein was recovered by centrifugation of the 50 ml tubes at 4500×g for 20 minutes. The supernatant was discarded. The protein pellets were washed by resuspension in 1 ml of ice cold HPLC-grade acetone and pelleted by centrifugation at 4500×g for 10 minutes. The acetone was decanted and the wash step was repeated using 1 ml of ice cold acetone. Protein was pelleted by centrifugation at 4500×g for 10 minutes. The wash step was repeated a third time and the final pellets were dried in a biological safety hood or with N₂ gas.

The final protein pellets were resuspended in 200 μl of Tris-HCl, pH 8.45. 10 μL of the suspension were combined with 10 μL of Laemmli buffer, heated at 70° C. for 10 minutes, and then loaded onto an SDS-PAGE gel.

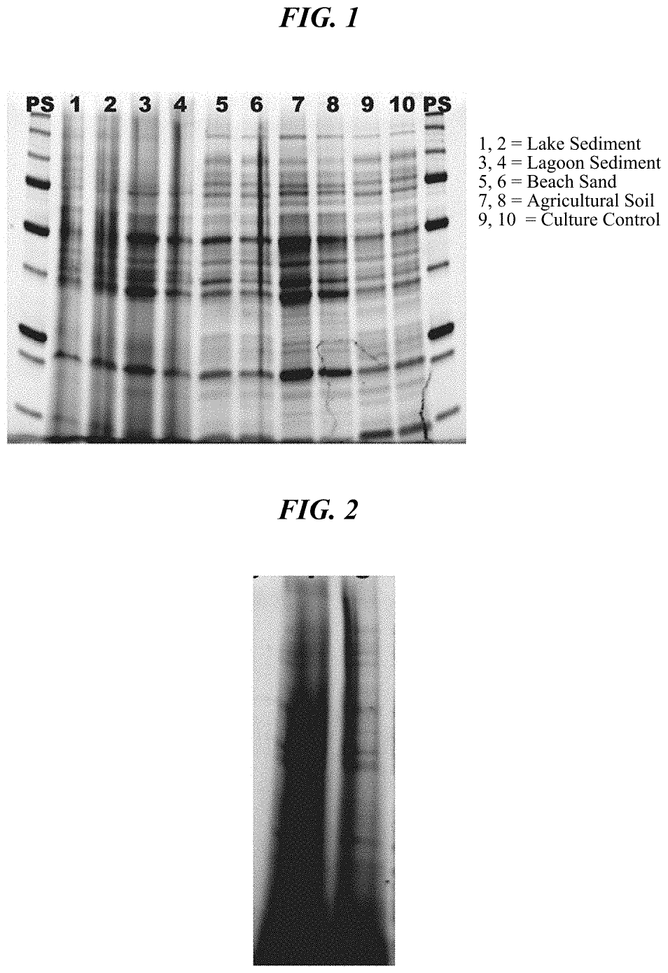

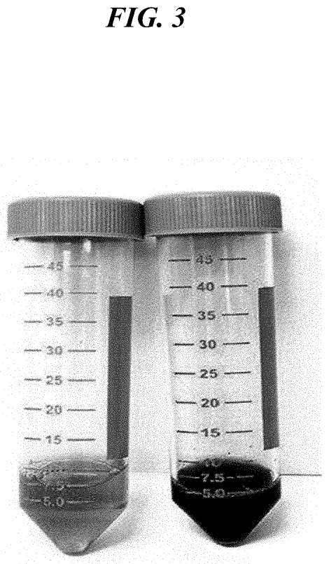

FIG. 1 shows the SDS-PAGE gel of the final protein collected from each sample, as well as an E. coli culture control sample. For comparison, FIG. 2 shows an SDS-PAGE gel of protein extracted from 5 g of E. coli-spiked soil using thermally assisted detergent-based cellular lysis with SDS, followed by TCA precipitation, as described by Chourey et al., (J. Proteome Res. 2010, 9(12): 6615-22). The gel in FIG. 2 has significant background staining, which can make it difficult to see the protein bands. In contrast, the bands in the gel of FIG. 1 have little to no background staining.

Modifications may be made to the foregoing without departing from the basic aspects of the methods and compositions provided herein. Although the compositions and methods have been described in substantial detail with reference to one or more specific embodiments, those of skill in the art will recognize that changes may be made to the embodiments specifically disclosed in this application, yet these modifications and improvements are within the scope and spirit of the methods and compositions provided herein.

All documents, including patents, patent application and publications cited herein, including all documents cited therein, tables, and drawings, are hereby expressly incorporated by reference in their entirety for all purposes.

While the methods and compositions have been described in detail with reference to certain Exemplary aspects thereof, it will be understood that modifications and variations are within the spirit and scope of that which is described and claimed.

1. A method for purifying or isolating a protein from a soil sample comprising cells and one or more humic substance contaminants, comprising:
(a) contacting the soil sample with a first solution comprising a non-ionic detergent, a buffer, one or more inorganic salts, and a polyol;
(b) contacting the resulting mixture of step (a) with a second solution comprising 1 to 6M inorganic salt and/or contacting the resulting mixture of step (a) with a second solution comprising an inorganic salt such that the total salt concentration in the mixture after the addition of the second solution is from 0.01 to 1M; and
(c) recovering the protein from the mixture.

2. The method of claim 1, wherein the first solution further comprises a chelating agent.

3. The method of claim 1, wherein the first solution further comprises an antifoaming agent.

4. The method of claim 1, wherein the inorganic salt in the second solution comprises NaCl.

5. The method of claim 1, further comprising contacting the sample with a disulfide-reducing agent following step (a).

6. The method of claim 1, further comprising one or both of:
(i) agitating the resulting mixture of step (a), and
(ii) agitating the resulting mixture of step (b).

7. The method of claim 6, wherein step (i), step (ii) or both steps (i) and (ii) are performed in the presence of beads.

8. The method according to claim 1, wherein the sample is agitated after addition of the first solution, optionally
(i) wherein agitation is carried out at room temperature or at a temperature below room temperature; and/or
(ii) wherein the sample is incubated at room temperature or at a temperature below room temperature prior to or following agitation.

9. The method according to claim 1, wherein the sample is agitated after addition of the second solution, optionally
(i) wherein agitation is carried out at room temperature or at a temperature below room temperature; and/or
(ii) wherein the sample is incubated at room temperature or at a temperature below room temperature prior to or following agitation.

10. The method according to claim 5, wherein the sample is agitated after addition of the disulfide-reducing agent, optionally
(i) wherein agitation is carried out at room temperature or at a temperature below room temperature; and/or
(ii) wherein the sample is incubated at room temperature or at a temperature below room temperature prior to or following agitation.

11. The method according to claim 1, further comprising one or both of:
(i) centrifuging the resulting mixture of step (a); and
(ii) centrifuging the resulting mixture of step (b).

12. The method according to claim 1, wherein the method comprises centrifuging the sample after addition of the second solution to separate undissolved material from the dissolved protein.

13. The method according to claim 1, wherein the protein is recovered from the solution by adding a precipitation agent to precipitate the protein out of the solution, optionally wherein the solution comprising the protein is obtained by removing undissolved material by filtration or centrifugation.

14. The method according to claim 1, wherein the first solution comprises:
(i) 0.01 to 2% by volume of non-ionic detergent;
(ii) 1 to 200 mM buffer, wherein optionally the buffer is a basic buffer;
(iii) 0.01 to 4 mM of a chelating agent;
(iv) 1 to 300 mM of one or more inorganic salts; and
(v) 1 to 30% by volume of a polyol; and/or
wherein the second solution is a 2 to 6M inorganic salt solution such that the total concentration of salt in the mixture after the addition of the second solution is 0.01 to 1M.

15. The method according to claim 1, wherein the first solution comprises Tris Base, EDTA, KCl, MgCl₂, glycerol, and Triton X-100; and wherein the second solution comprises NaCl.

16. The method of claim 1, comprising one or both of:
(i) agitating the resulting mixture of step (a) in the presence of ceramic beads, glass beads or a mixture thereof; and
(ii) agitating the resulting mixture of step (b) in the presence of ceramic beads, glass beads or a mixture thereof.

17. The method of claim 1, wherein the soil sample comprises cells, wherein
the first solution comprises Tris Base, EDTA, KCl, MgCl₂, glycerol, and Triton X-100,
the inorganic salt of the second solution is sodium chloride, and
step (c) comprises removing the one or more humic substance contaminants.

18. The method according to claim 1, wherein the first solution comprises 10 to 100 mM Tris Base, 0.1 to 2 mM EDTA, 10 to 100 mM KCl, 10 to 100 mM MgCl₂, 5 to 20% by volume glycerol, and 0.1 to 1% by volume Triton X-100.

19. The method according to claim 18, wherein the first solution contains about 20 mM Tris base, about 2 mM EDTA, about 10 mM KCl, about 10 mM MgCl₂, about 10% by volume glycerol, and about 1% Triton X-100.

20. The method according to claim 1, wherein the second solution comprises 2 to 5M or 4 to 4.5M NaCl.

21. The method according to claim 1, wherein the soil sample is selected from sediment, manure, compost, dust, dirt, mud, ground, landfill and mulch and/or the one or more humic substances are selected from humic acids and fulvic acids.

22. A method for removing one or more humic substance contaminants from a soil sample, comprising:
(a) contacting the soil sample with a first solution comprising a non-ionic detergent, a buffer, one or more inorganic salts, and a polyol;
(b) contacting the resulting mixture of step (a) with a second solution comprising 1 to 6M inorganic salt and/or contacting the resulting mixture of step (a) with a second solution comprising an inorganic salt such that the total salt concentration in the mixture after the addition of the second solution is from 0.01 to 1M; and
(c) removing the one or more humic substance contaminants.

23. The method according to claim 22, further comprising obtaining a solution containing a dissolved protein and recovering the protein from the solution.
